(19) United States
(12) Patent Application Publication
(10) Pub. No.: US 2005/0178389 A1
(43) Pub. Date: Aug. 18, 2005

(54) Disease indications for selective endobronchial lung region isolation

(76) Inventors: David Peter Shaw, Christchurch (NZ); John McCutcheon, Menlo Park, CA (US); Antony J. Fields, San Francisco, CA (US)

Correspondence Address: FISH & RICHARDSON, PC, 12390 EL CAMINO REAL, SAN DIEGO, CA, 92130-2081, US

(21) Appl. No.: 11/044,875

(22) Filed: Jan. 26, 2005

Related U.S. Application Data
(60) Provisional application No. 60/539,671, filed on Jan. 27, 2004.

(51) Int. Cl. A61M 11/00; A61M 16/00

(52) U.S. Cl. 128/207.15

(57) ABSTRACT

Disclosed are various disease indications and treatment methods that benefit from selective lung region isolation. A lung region is bronchially isolated by regulating the flow of fluid to and from the lung region, such as by implanting one or more bronchial isolation devices into one or more bronchial passageways that feed air to the lung region. The bronchial isolation devices can comprise, for example, one-way valves, two-way valves, occluders or blockers, ligating clips, glues, sealants, and sclerosing agents.

### RELATED APPLICATIONS

This application claims the benefit of priority to U.S. Provisional Patent Application Ser. No. 60/539,671, entitled “Disease Indications For Selective Endobronchial Lung Region Isolation”, filed Jan. 27, 2004, which is incorporated herein by reference in its entirety.

### BACKGROUND

Various devices can be used to achieve the bronchial isolation of one or more selected regions of the lung. Pursuant to a lung region bronchial isolation process, at least one flow control device (also referred to as a bronchial isolation device) is implanted within one or more bronchial passageways that provide fluid flow to and from the lung region to thereby “isolate” the lung region. The lung region is isolated in that fluid flow to and from the lung region is regulated or blocked through the bronchial passageway(s) in which the device is implanted. For example, the flow of fluid (gas or liquid) past the device in the inhalation direction can be prevented while allowing flow of fluid in the exhalation direction, or the flow of fluid past the implanted device in both the inhalation and exhalation directions can be prevented. The flow control devices can comprise, for example, one-way valves, two-way valves, occluders or blockers, ligating clips, glues, sealants, sclerosing agents, etc.

One common feature of lung region flow control devices (such as, for example, one-way valves, two-way valves, occluders or blockers, ligating clips, glues, sealants, sclerosing agents, etc.) and corresponding techniques is that they prevent or substantially inhibit the flow of fluid (gas or liquid) past the device in the inhalation direction, thus isolating the lung region distal to the device. It has been determined that selective lung region isolation is effective in treating pulmonary emphysema. However, there is a need for the identification of other diseases and conditions that would benefit from selective lung region isolation.

### SUMMARY

Disclosed are various disease indications and treatment methods that benefit from selective lung region isolation. In one aspect, there is disclosed a method of treating pulmonary hypertension in a human or mammal comprising blocking fluid flow in a bronchial passageway sufficiently to reduce pulmonary hypertension.

In another aspect, there is disclosed a method of treating pulmonary hypertension in a human or mammal comprising: assessing a level of pulmonary hypertension of a patient; and reducing fluid flow into a selected region of a lung until pulmonary hypertension is reduced.

In another aspect, there is disclosed a method of reducing pulmonary hypertension in a patient comprising: assessing pulmonary function; comparing the pulmonary function to an eligibility threshold; and if pulmonary function is higher than the eligibility threshold, blocking fluid flow into a selected region of the lung, wherein pulmonary hypertension is reduced.

In another aspect, there is disclosed a method of improving lung function of a patient comprising: measuring a lung function indicator to obtain an initial value; comparing the initial value to a threshold value; and if the initial value is higher than the threshold value, blocking fluid flow into one or more regions of the lung sufficiently to raise the lung function indicator above the initial value.

In yet another aspect, there is disclosed a method of treating low carbon monoxide diffusing capacity of a lung (DLCO) in a patient comprising: measuring an initial DLCO; comparing the initial DLCO to a threshold DLCO; and if the initial DLCO is higher than the threshold DLCO, blocking fluid flow into one or more regions of the lung sufficiently to achieve an increase in DLCO.

In yet another aspect, there is disclosed a method of treating low carbon monoxide diffusing capacity of a lung (DLCO) in a patient comprising blocking fluid flow into one or more regions of the lung to achieve an increase in DLCO without collapsing or removing the regions of the lung.

In yet another aspect, there is disclosed a method of treating tuberculosis, comprising: bronchially isolating a lung region to reduce the delivery of oxygen to the lung region and deprive M. tuberculosis bacillus of oxygen in the lung region; and, in combination with bronchially isolating the lung region, administering a chemotherapeutic drug to the lung region.

In yet another aspect, there is disclosed a method of treating an air leak in a lung of a patient, comprising: identifying at least one bronchial passageway that provides airflow to a region of the lung that contains the air leak; and blocking fluid flow through the identified bronchial passageway.

Other features and advantages should be apparent from the following description of various embodiments, which illustrate, by way of example, the principles of the disclosed devices and methods.

### BRIEF DESCRIPTION OF THE DRAWINGS

FIG. 1 shows an anterior view of a pair of human lungs and a bronchial tree with a bronchial isolation device implanted in a bronchial passageway to bronchially isolate a region of the lung.

FIG. 2 shows a perspective view of an embodiment of a bronchial isolation device.

FIG. 3 shows a cross-sectional view of the device of FIG. 2.

### DETAILED DESCRIPTION

There is now described exemplary devices and methods for bronchially isolating a region of the lung. A lung region is bronchially isolated by regulating the flow of fluid to and from the lung region, such as by implanting one or more bronchial isolation devices into one or more bronchial passageways that feed air to the lung region. The bronchial isolation devices can comprise, for example, one-way valves, two-way valves, occluders or blockers, ligating clips, glues, sealants, sclerosing agents, etc. The regulation of the flow of fluid can include blocking the flow of fluid in one direction while permitting flow in another direction or blocking fluid flow in both directions through the bronchial passageway. The flow of fluid can also be substantially inhibited in one or both directions.

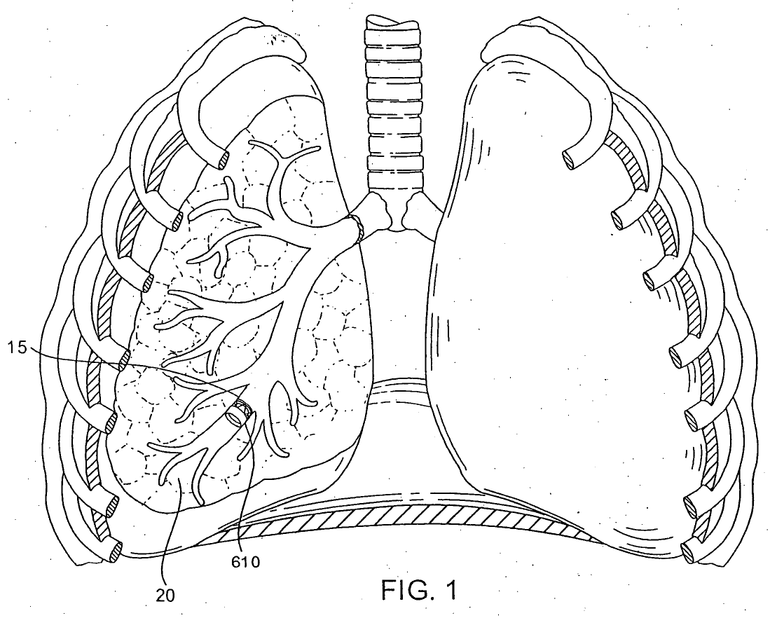

As shown in FIG. 1, in one exemplary embodiment, the bronchial isolation of the targeted lung region is accomplished by implanting a blocking element, such as a flow control device comprising a bronchial isolation device 610, into the lung. The device 610 is implanted into a bronchial passageway 15 that feeds air to a targeted lung region 20. The bronchial isolation device 610 regulates airflow through the bronchial passageway 15, such as by permitting fluid flow in one direction (e.g., the exhalation direction) while limiting or preventing fluid flow in another direction (e.g., the inhalation direction).

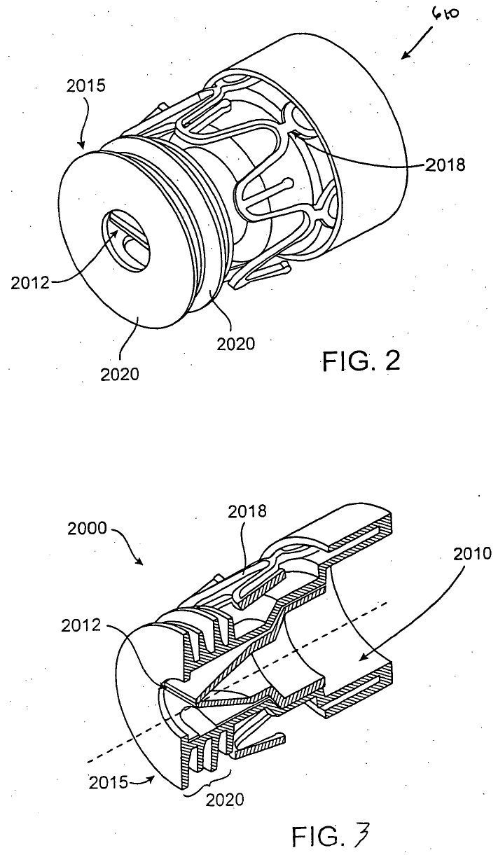

FIGS. 2 and 3 show an exemplary bronchial isolation device 610 that can be used to achieve one-way flow. The flow control element 610 includes a main body that defines an interior lumen 2010 through which fluid can flow along a flow path. The flow of fluid through the interior lumen 2010 is controlled by a valve member 2012. The valve member 2112 in FIGS. 2-3 is a one-way valve, although two-way valves can also be used, depending on the type of flow regulation desired.

With reference still to FIGS. 2-3, the bronchial isolation device 610 has a general outer shape and contour that permits the flow control bronchial isolation device to fit entirely within a body passageway, such as within a bronchial passageway. The bronchial isolation device 610 includes an outer seal member 2015 that provides a seal with the internal walls of a body passageway when the flow control device is implanted into the body passageway. The seal member 2015 includes a series of radially-extending, circular flanges 2020 that surround the outer circumference of the flow control device 610. The bronchial isolation device 610 also includes an anchor member 2018 that functions to anchor the bronchial isolation device 610 within a body passageway.

The following references describe exemplary bronchial isolation devices and delivery devices: U.S. Pat. No. 5,954,766 entitled “Body Fluid Flow Control Device”; U.S. patent application Ser. No. 09/797,910, entitled “Methods and Devices for Use in Performing Pulmonary Procedures”; U.S. patent application Ser. No. 10/270,792, entitled “Bronchial Flow Control Devices and Methods of Use”; U.S. patent application Ser. No. 10/448,154, entitled “Guidewire Delivery of Implantable Bronchial Isolation Devices in Accordance with Lung Treatment”; and U.S. patent application Ser. No. 10/275,995, entitled “Bronchiopulmonary Occlusion Devices and Lung Volume Reduction Methods”. The foregoing references are all incorporated by reference in their entirety and are all assigned to Emphasys Medical, Inc., the assignee of the instant application. It should be appreciated that other types of bronchial isolation devices can be used.

There are at least two possible effects of selective lung region isolation. One such effect is that the isolated lung region collapses and becomes atelectatic either quickly or over an extended period of time. Another possible effect is that the isolated lung region does not collapse (due to collateral ventilation to the lung region or for other reasons). In both cases, inhaled air is prevented or substantially inhibited from flowing into the isolated lung region through the bronchial lumens in which the bronchial isolation device is implanted. The inhaled air is thus preferentially redirected to non-isolated regions of the lung. It has been determined that numerous diseases and conditions can benefit from selective lung region isolation. At least some of these diseases and conditions are listed and described herein.

1. Tuberculosis

The term tuberculosis (TB) describes an infectious disease that is caused by two species of mycobacterium: M. bovis and M. tuberculosis. M. bovis infects mainly cattle. M. tuberculosis is a strict aerobe, and an anaerobic environment effectively inhibits mycobacterial growth. It was discovered that patients who developed a pneumothorax with pulmonary tuberculosis frequently had an improvement in their symptoms. This observation led to the concept of therapeutic artificially induced pneumothorax (TAIP). TAIP gained popularity as a treatment method during the beginning of the 20th Century and it required repeated installation of gas into the pleural space at 2-3 week intervals. The effect of the TAIP is to collapse an entire lung with decreased ventilation to this lung associated with the physiological hypoxic vasoconstriction of the pulmonary vasculature. The mycobacterium was thus starved of oxygen.

There are a variety of methods for collapsing the lung, such as crushing of the phrenic nerve and pneumoperitoneum. Unfortunately, these latter two procedures tend to collapse the lower lobes predominantly, which is a less desirable outcome because tuberculosis affects predominantly the upper lobe. In order to address this problem, various ingenious surgical techniques, such as the placement of ping-pong ball-like, space-occupying lesions into the upper hemi-thorax were introduced, with the objective of selectively collapsing the upper lobe. Surprisingly good results were achieved with this therapy with sputum conversion in 30-60% of patients. The use of such a therapy was in the setting of an era before antituberculous drugs.

The mainstay of current TB treatment is antituberculous drugs. Such drugs were first introduced in the early 1940's and have become so effective that surgical techniques are largely considered obsolete. It was discovered early in the treatment of TB that drug-resistant strains would emerge quickly if a patient was treated with just one agent. To avoid the emergence of drug-resistant strains, 2 or 3 agents are used concurrently during treatment, with typical treatment being measured in months and up to years in some cases. The first line drugs that are typically used include: isoniazid, streptomycin, rifampin, ethambutol, thiacetazone, and aminosalicylate sodium. The second line drugs that are used include: ethionamide, cycloserine, kanamycin, and capreomycin. Current drug dosing regimes are typically 4 drugs for 2 months followed by 2 drugs for 4 months.

Not unexpectedly, multi-drug resistant strains have emerged. The phenomena of multi-drug resistant strains is making the treatment of TB very difficult in some areas. This is a problem that is likely to get worse rather than better. Additionally, tuberculosis is now featuring prominently in the disease process of patients who are immunosuppressed, e.g. AIDS patients. Traditionally, TB has been associated with poor socioeconomic conditions. To a large extent, TB is still a current problem with the immigrant population in developed countries such as the United States. Moreover, the need for long therapeutic courses and poor compliance make the treatment of TB very difficult. Physicians in the United States have taken to programs of having TB infected immigrants visit the doctor's office on a daily basis and have them supervised while they swallow their pills.

It has been determined that a portion of the lung can be endobronchially isolated pursuant to a TB treatment regimen with beneficial results. If the portion of the lung infected with M. tuberculosis (typically the upper lobe) is isolated endobronchially using any of the previously-mentioned lung region isolation techniques, the air flow in the inhalation direction to the isolated lung tissue is minimized or eliminated. The reduction or elimination of air flow to the isolated lung tissue reduces or eliminates the delivery of oxygen to isolated tissues and deprives the M. tuberculosis bacillus of oxygen. In addition, there is likely associated hypoxic vasoconstriction in the blood vessels in the isolated lung tissue, which further reduces the potential of oxygen delivery to the isolated lung tissues. This oxygen deprivation can be lethal to the bacillus; however, for completeness it can be desirable to combine such bronchial isolation therapy with the administration of current chemotherapeutic drugs such as isoniazid and rifampin etc.

Thus, pursuant to a TB treatment method, a region of the lung is bronchially isolated, such as by implanting one or more bronchial isolation devices into a bronchial passageway that feeds fluid to the lung region. The bronchial isolation may be combined with the administration of chemotherapeutic drugs such as isoniazid and rifampin etc.

The combination of this two pronged approach (selective lung region isolation and drug therapy) potentially has many benefits. One such benefit is that the chemotherapeutic agents are more lethal to organisms that are oxygen-deprived. This is important in preventing the emergence of drug-resistant strains. In addition, prolonged antibacterial courses can be shortened; this has beneficial implications for both cost and patient compliance issues. A shorter antimicrobial course also has potential advantages from a drug toxicity point of view with the potential for reduction of adverse side effects (e.g. ethambutol can cause blindness with prolonged treatment). Moreover, the technique of selective lung region isolation has a much lower morbidity than many surgical techniques and is applicable to both sides of the lung simultaneously. In contrast, the surgically-induced pneumothorax procedures can be performed on one side of the lung only. The addition of endobronchial lung region isolation to the standard drug therapy for TB has the potential to decrease the 6 month drug course, and/or reduce the number of drugs thus reducing the side effect of the drugs and increasing compliance.

2. Pulmonary Hypertension

The technique of lung region isolation can also be used beneficially in the treatment of pulmonary hypertension. Pulmonary hypertension is defined as abnormally elevated blood pressure in the pulmonary circuit. The pulmonary hypertension may be primary, or secondary to pulmonary or cardiac disease (such as fibrosis of the lung or mitral stenosis). There are a number of types of pulmonary hypertension, and some are described as follows:

1. Arterial Pulmonary Hypertension: In this class of hypertension the pulmonary circuit is subjected to elevated pressures due to pathology such as a ventricular septal defect. This leads to irreversible changes in the small pulmonary arterial vessels that further leads to a raised peripheral vascular resistance. This results in a rise of the pressure in the pulmonary arterial circuit.

2. Chronic Thromboemboli: In this condition, thrombi are thrown off and deposited in the lungs. Over time these emboli become organized and form a layer on the inside of the arterial vessels, which then results in a rise in the blood pressure due to the increase resistance to blood flow in the occluded vessels.

3. Post-Capillary Pulmonary Hypertension: In this class of pulmonary hypertension, a high back-pressure is created across the vasculature of the lungs as the result of pathology in the left side of the heart. One example is when there is mitral valve incompetence that raises the left atrial pressure, which in turn increases the back pressure across the lungs. This results in a rise in pulmonary pressure that is necessary in order to transport the blood through the lungs. Repairing or replacing the mitral valve can reduce the pulmonary pressures by 50% in a surprisingly short period of time.

4. Extrinsic Vascular Compression: In this class of pulmonary hypertension, blood pressure in the lungs rises due to restrictions in the blood vessels in the lungs arising from extrinsic compression of the blood vessels. Extrinsic compression of the blood vessels can arise from a number of conditions, including emphysema. In emphysema, diseased portions of the lung can become hyperinflated due to loss of elastic recoil, and these regions can compress the non-diseased portions of the lung. The compression can, in turn, compress the vasculature leading to pulmonary hypertension.

The fourth class of pulmonary hypertension listed above, extrinsic vascular compression, can be helped greatly through the isolation of selected portions of the lung. In particular, if the hyperinflated regions of the lung are isolated through the implantation of one or more bronchial isolation devices in one or more bronchial passageways that lead to the hyperinflated lung region(s). The regions are either reduced in volume or are completely collapsed as a result of the isolation This reduction in the volume of these regions reduces the extrinsic compression of the pulmonary vasculature, and results in a reduction in blood pressure and thus in pulmonary hypertension. Pulmonary hypertension is often seen in patients with chronic obstructive pulmonary disease (COPD), and especially in patients with emphysema (a disease that is a subset of COPD) and this condition can be treated with implanted bronchial isolation devices.

Thus, hypertension can be treated pursuant to a method of bronchially isolating one or more lung regions. A method of treating pulmonary hypertension in a human or mammal comprises, for example, delivering a therapeutically effective quantity of a fluid-blocking material to one or more bronchial passageways to reduce pulmonary hypertension. Such methods can include delivering a therapeutically effective quantity of a fluid-blocking material to one or more bronchial passageways to reduce pulmonary hypertension, as well as blocking fluid flow in a bronchial passageway sufficiently to reduce pulmonary hypertension. In another method, a level of pulmonary hypertension of a patient is assessed, and fluid flow through a lung passageway is blocked until the level of pulmonary hypertension decreases. Thus, fluid flow into a selected region of a lung is reduced until pulmonary hypertension is reduced.

The bronchial isolation process can include redirecting fluid flow away from a selected region of a lung until pulmonary hypertension is reduced. The fluid flow can be redirected or blocked by placing a blocking element in a bronchial passageway communicating with the lung region, wherein the blocking element inhibits fluid flow into the target lung region without collapsing the target lung region. Alternately, the lung region can be collapsed.

Pursuant to another method of reducing pulmonary hypertension in a patient, a pulmonary function is assessed and compared to an eligibility threshold. In one embodiment, for example, the eligibility threshold is the maximum pulmonary function with which the patient is suitable for lung volume reduction. If the assessed pulmonary function is higher than the eligibility threshold, fluid flow into a selected region of the lung is blocked or otherwise regulated to reduce pulmonary hypertension.

3. Obstructive Lung Diseases

The use of selective endobronchial lung region isolation for the treatment of emphysema has been previously disclosed. However, there are other obstructive lung diseases (aside from emphysema) that may be successfully treated with selective lung region isolation.

Chronic bronchitis is also an obstructive disease, though the obstruction is in the more proximal airways rather than in the most distal airways, as is the case with emphysema. Given this, a patient with chronic bronchitis benefits from selective isolation of the most diseased regions of their lungs. Pursuant to this treatment method, one or more bronchial isolation devices are implanted in a bronchial passageway that feeds air to the diseased regions. This results in inhaled air being redirected away from the isolated lung regions and towards the healthier, non-isolated lung regions, resulting in improved pulmonary function.

Obliterative bronchiolitis or bronchiolitis obliterans is another obstructive disease that benefits from treatment with selective lung region isolation. The disease is often accompanied by hyperinflated lung regions in the most diseased areas, but not always. If the most diseased lung regions are isolated using any of the previously mentioned selective lung isolation techniques, the inhaled air is redirected to other healthier, non-isolated regions of the lung, and thus improving overall pulmonary function.

4. Treatment of Ventilation/Perfusion Mismatch

Lung region isolation can also be used as a treatment for ventilation/perfusion mismatch. There are numerous conditions and diseases that result in a ventilation/perfusion mismatch or shunt. In this condition, there is insufficient ventilation to portions of the lung, with the result that poorly oxygenated blood is returned to the arterial system of the body. This leads to hypoxemia. Selectively isolating the regions of the lung that are poorly ventilated results in improved lung function in the remaining non-isolated lung regions. This benefit can occur both if the isolated lung region is collapsed and if it is not.

In either case, inhaled air is preferentially redirected to the healthier, non-isolated lung regions through the implantation of one or more bronchial isolation devices in the appropriate bronchial passageway(s) of the lung. The reduction or elimination of inhaled oxygen to the isolated lung region induces hypoxic vasoconstriction in the blood vessels of the isolated lung region. This reduces the blood flow to the isolated lung region and thus improves ventilation/perfusion matching. In addition, as a result of redirection of airflow, ventilation increases to the non-isolated lung regions, thus improving pulmonary function. Some of the diseases that benefit from treatment of this sort are acute respiratory distress syndrome (ARDS), and pulmonary embolism.

5. Treatment of Low Diffusing Capacity (DLco)

There are a number of diseases of the lung that can result in a reduced carbon monoxide diffusing capacity (DLco). Diffusing capacity is a measure of the lung's ability to transfer oxygen to the blood flowing through the pulmonary vessels, and often results in low oxygen saturation and hypoxemia. DLco can be pathologically low due to many different disease states including emphysema and chronic bronchitis. Treatment for low DLco may take a number of different forms.

A primary method of treating low DLco is to perform selective lung region isolation on the regions that are most effected by the particular disease that is present. In the case of emphysema, for example, the areas of greatest parenchymal destruction as determined by CT scan are targeted for selective lung region isolation. In the case of chronic bronchitis, the areas of greatest obstruction to airflow is targeted. As mentioned, selective lung region isolation can be accomplished by implanting one or more bronchial isolation devices (e.g., one-way valves, two-way valves, occluders or blockers, ligating clips, glues, sealants, sclerosing agents, etc) into one or more bronchial passageways that feed fluid to the lung region. Once selective lung region isolation is performed, inhaled air is blocked to the isolated regions, and inhaled air is redirected to other healthier, non-isolated regions of the lung. This results in an improvement of overall pulmonary function.

A second method for treating low DLco is to determine the areas of the lung that have the lowest DLco, and to perform selective lung region isolation on these areas. That is, one or more bronchial isolation devices are implanted into one or more of the bronchial passageways that feed air to the areas of the lung with the lowest DLco. Existing methods for measuring DLco are performed on the whole lung. Thus, existing methods often do not identify the regions that have the lowest diffusing capacity. Selectively performing diffusing capacity tests on sub-sections of the lung (such as a lobe or a segment) allows the region of lowest DLco to be determined and treated with selective lung region isolation.

Existing tests that are of use in determining regions of low DLco are the ventilation and perfusion scans. In addition, there are nuclear imaging techniques that identify regions of the lung that have poor ventilation or perfusion. Regions that have poor ventilation, poor perfusion or both are regions that are highly likely to correspond to regions of low DLco. Thus if these regions are treated with selective lung region isolation, inhaled air is blocked to the isolated regions, inhaled air is redirected to other healthier non-isolated regions of the lung, and overall pulmonary function is improved.

Thus, pursuant to a method of treating low carbon monoxide diffusing capacity of a lung (DLCO) in a patient, an initial DLCO is measured. The initial DLCO is then compared to a threshold DLCO. The threshold DLCO is the maximum DLCO with which the patient is eligible for lung volume reduction. If the initial DLCO is higher than the threshold DLCO, fluid flow into one or more regions of the lung is blocked or substantially inhibited sufficiently to achieve an increase in DLCO. The method of treating low carbon monoxide diffusing capacity of a lung (DLCO) can comprise blocking fluid flow into one or more regions of the lung to achieve an increase in DLCO without collapsing or removing the regions of the lung.

6. Treatment of Air Leaks in the Lung

There are a number of situations where air can leak from the lung through a pathway other than through normal pathways of respiration. That is, a pathway exists that permits the movement of air either into or out of the lung or both, wherein the pathway does not comprise the bronchial tree and the trachea. Such air leaks can take different forms and can be caused by different events and diseases. There is now described some exemplary events and diseases that can cause lung air leaks through pathways other than the bronchial tree and trachea.

Bronchopleural Fistula

Bronchopleural fistula (BPF) is an open air connection between the bronchial tree and the pleural space of the lung.

Lung Air Leak

A lung air leak is defined as a connection between the alveolar space and the pleural space, or between a bleb or bullae and the pleural space.

A pneumothorax is defined as the presence of free air between the visceral and parietal pleura. It is appreciated that both BPFs and air leaks will almost always result in a pneumothorax. A pneumothorax, however, can result in the absence of a lung air leak or a BPF when there is a penetrating injury to the chest wall without the lung being injured. Both BPFs and air leaks can be caused by a number of different pathologies including, for example:
Trauma, such as a puncture wound through the chest wall;
Latrogenic causes such as due to chest aspiration, intercostal nerve block, transbronchial biopsy, needle aspiration lung biopsy, positive pressure ventilation, subclavian cannulation, etc.;
Chest compression injury including external cardiac massage;
Secondary to surgical interventions such as lung resection, etc.;
Spontaneous pneumothorax;
Secondary to degenerative lung diseases such as emphysema, COPD, asthma, etc.;
Secondary to inflammatory or infective diseases such as AIDS, vasculitis, cystic fibrosis, lung abscess, tuberculosis, whooping cough, sarcoidosis, etc.;
Secondary to other diseases such as congential cysts and bullae, etc.;

Regardless of the specific cause of the air leak, it is essential for normal functioning of the lungs to close and seal the air leak or BPF.

Treatment of Air Leak

In all cases of air leaks (such as those described above), there is an uncontrolled loss of air from the lung, which usually results in a pneumothorax. The currently accepted treatments for air leaks and BPFs include:
Rest and oxygen therapy;
Needle aspiration of the air;
Simple intercostal drainage with or without vacuum;
Medical thoracoscopy with talc poudrage;
Video-assisted thoracic surgery (VATS) with pleural abrasion or partial pleurectomy and bullectomy;
Thoracotomy or medial sternotomy with surgical repair;
Fibrin or other glue injection into bronchus leading to air leak or BPF;

Many pneumothoraces will heal with one or more of these interventions. However some will not, and often it is difficult or impossible for a patient to tolerate some of the more invasive interventions such as surgical repair. Even simple intercostals drainage requires creating an opening into the chest cavity and the insertion of a chest tube for drainage.

What is needed is a simple and minimally invasive method of blocking air loss from the lungs as a result of an air leak. In addition, it would be beneficial if the intervention could be reversed or removed once the lung has had a chance to heal. There is now disclosed such a method.

If the bronchus that feeds the air leak is identified, one or more bronchial isolation device can implanted in one or more bronchial passageways to isolate the region of the lung that contains the air leak or BPF. The device(s) would prevent further air flow through the leak site (i.e., through the bronchus that feeds the air leak). The bronchial isolation device can be removable, such that the device can be removed from the lungs once the air leak or fistula had healed. Once removed, normal air flow through the bronchial passageway is restored and the isolated lung tissue can return to functionality.

As mentioned previously, the implanted bronchial isolation device may be a blocker that prevents the flow of liquid (such as mucus) or gas (such as air) in both the inhalation and the exhalation direction. These devices could include, for example, plugs or occluders, glues, ligating clips, etc. Once implanted, the device(s) prevent air from flowing through the bronchial lumen and out of the lung through the air leak or BPF location. In one embodiment, the bronchial isolation device comprises a removable one-way valve device that prevent gas from flowing into the isolated lung region yet allows gas and mucus to escape naturally in the exhalation direction in a way that a blocking device would not.

Identification of Leak Location

Pursuant to one step in a method of treating an air leak, one or more bronchial lumens that feed the air leak are identified. It is critical to correctly identify the bronchial lumen or lumens that feed the site of the air leak in order to determine the optimal placement location for the bronchial isolation device(s). If the patient is already on a chest drain, there will normally be air bubbling through the water valve (if a water valve is used) or air venting through the Heimlich valve (if a Heimlich valve is used) of the drain. The leak source may be readily identified by inserting a bronchoscope (rigid or flexible) into the bronchial tree of the patient, and inserting a flexible balloon catheter into the working channel of the scope. The balloon is then inserted and inflated into each bronchial lumen in turn. When the correct bronchial lumen is blocked using the flexible balloon catheter, bubbling through the water valve or venting through the Heimlich valve will stop. Given that it would be advantageous to isolate the smallest amount of lung possible while still stopping the leak, the bronchial isolation device can be implanted in the most distal branch possible after the lumen is identified. In some situations, the air leak may be fed by more than one bronchial lumen. In these cases, bronchial isolation devices are implanted in all bronchial lumens that feed the air leak.

If the patient does not have a chest drain, the leak site may be identified other ways. One method is to inject a small amount of a visible dye, such as, for example, methylene blue, into the pleural space of the suspect lung. If the bronchial tree is monitored visually with a bronchoscope while the patient coughs, the source bronchial lumen can be found by looking for expectorated blue dye.

An alternative method is to inject a small amount of radiographic contrast into the pleural space of the suspect lung and monitoring the progress of the contrast with fluoroscopy or on CT scan during cough and normal breathing. If a flexible bronchoscope is inserted into the bronchial tree during fluoroscopy, it may be guided to the bronchus that is expectorating the radiographic contrast, and thus the bronchus leading to the air leak or BPF may be identified. Once identified, the lung portion that contains the air leak can be isolated by implanting a bronchial isolation device or devices into the appropriate bronchial passageway. As before, the device or devices can be implanted as distally as possible in order to isolate the minimal amount of lung tissue.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A method of treating pulmonary hypertension in a human or mammal comprising blocking fluid flow in a bronchial passageway sufficiently to reduce pulmonary hypertension.

2. The method of claim 1, wherein blocking fluid flow in a bronchial passageway sufficiently to reduce pulmonary hypertension comprises delivering a therapeutically effective quantity of a fluid-blocking material to one or more bronchial passageways to reduce pulmonary hypertension.

3. A method of treating pulmonary hypertension in a human or mammal comprising:
assessing a level of pulmonary hypertension of a patient; and
reducing fluid flow into a selected region of a lung until pulmonary hypertension is reduced.

4. The method of claim 3, wherein reducing fluid flow into a selected region of a lung until pulmonary hypertension is reduced comprises blocking fluid flow through a lung passageway until the level of pulmonary hypertension decreases.

5. The method of claim 3, wherein reducing fluid flow into a selected region of a lung until pulmonary hypertension is reduced comprises redirecting fluid flow away from a selected region of a lung until pulmonary hypertension is reduced.

6. The method of claim 3, wherein reducing fluid flow into a selected region of a lung until pulmonary hypertension is reduced comprises placing a blocking element in a bronchial passageway communicating with the lung region, the blocking element inhibiting fluid flow into the lung region without collapsing the target lung region.

7. A method of reducing pulmonary hypertension in a patient comprising:
assessing pulmonary function;
comparing the pulmonary function to an eligibility threshold; and
if pulmonary function is higher than the eligibility threshold, blocking fluid flow into a selected region of the lung;
wherein pulmonary hypertension is reduced.

8. A method of improving lung function of a patient comprising:
measuring a lung function indicator to obtain an initial value;
comparing the initial value to a threshold value; and
if the initial value is higher than the threshold value, blocking fluid flow into one or more regions of the lung sufficiently to raise the lung function indicator above the initial value.

9. A method of treating low carbon monoxide diffusing capacity of a lung (DLCO) in a patient comprising:
measuring an initial DLCO;
comparing the initial DLCO to a threshold DLCO; and
if the initial DLCO is higher than the threshold DLCO, blocking fluid flow into one or more regions of the lung sufficiently to achieve an increase in DLCO.

10. A method of treating low carbon monoxide diffusing capacity of a lung (DLCO) in a patient comprising blocking fluid flow into one or more regions of the lung to achieve an increase in DLCO without collapsing or removing the regions of the lung.

11. A method of treating tuberculosis, comprising:
bronchially isolating a lung region to reduce the delivery of oxygen to the lung region and deprive M. tuberculosis bacillus of oxygen in the lung region; and
in combination with bronchially isolating the lung region, administering a chemotherapeutic drug to the lung region.

12. The method of claim 12, wherein the chemotherapeutic drug comprises isoniazid or rifampin.

13. The method of claim 12, wherein bronchially isolating a lung region comprises implanting one or more bronchial isolation devices into a bronchial passageway that feeds fluid to the lung region.

14. The method of claim 12, wherein the bronchial isolation device comprises a one-way valve device that prevent gas from flowing in an inhalation direction and permits gas and mucus to flow in an exhalation direction.

15. A method of treating an air leak in a lung of a patient, comprising:
identifying at least one bronchial passageway that provides airflow to a region of the lung that contains the air leak;
blocking fluid flow through the identified bronchial passageway.

16. The method of claim 15, wherein blocking fluid flow through the identified bronchial passageway comprises implanting one or more bronchial isolation devices into at least one bronchial passageway that provides airflow to the region of the lung.

17. The method of claim 16, wherein the bronchial isolation device comprises a one-way valve.

18. The method of claim 15, wherein blocking fluid flow through the identified bronchial passageway comprises blocking fluid flow through a plurality bronchial passageways that provides airflow to the region of the lung.

19. The method of claim 15, wherein identifying at least one bronchial passageway comprises using a balloon catheter to successively block airflow through bronchial passageways that provide airflow to the region of the lung until an indication is observed that air is no longer flowing through the chest drain.

20. The method of claim 15, wherein identifying at least one bronchial passageway comprises:
injecting a visible dye into a pleural space of the lung;
monitoring the bronchial passageways of the lung for expectoration of dye.

21. The method of claim 20, wherein the monitoring step is performed while the patient coughs.

22. The method of claim 15, wherein identifying at least one bronchial passageway comprises:
injecting a radiographic contrast into a pleural space of the lung;
monitoring movement of the contrast through the lung with fluoroscopy or on CT scan during cough and normal breathing of the patient.
